(19) United States
(12) Patent Application Publication
(10) Pub. No.: US 2009/0041891 A1
(43) Pub. Date: Feb. 12, 2009

(54) Gene Encoding Transcriptional Inducer for Maltase Gene and Maltose Transporter Gene and use Thereof

(75) Inventors: Yoshihiro Nakao, Osaka (JP); Yukiko Kodama, Osaka (JP); Tomoko Shimonaga, Osaka (JP)

Correspondence Address: DRINKER BIDDLE & REATH (DC), 1500 K STREET, N.W., SUITE 1100, WASHINGTON, DC, 20005-1209, US

(73) Assignee: SUNTORY LIMITED, Osaka-shi, Osaka (JP)

(21) Appl. No.: 12/280,805

(22) PCT Filed: Feb. 21, 2007
(86) PCT No.: PCT/JP2007/053706

§ 371 Date: Aug. 27, 2008

(30) Foreign Application Priority Data
Mar. 1, 2006 (JP) 2006-055542

(51) Int. Cl. C12C 11/00; C12N 15/11; C07K 14/00; C12Q 1/68; C12N 15/00; C12N 1/19

(52) U.S. Cl. 426/16; 536/23.5; 530/350; 435/320.1; 435/254.2; 426/11; 426/592; 435/6

(57) ABSTRACT

The present invention relates to a gene encoding a transcriptional inducer for maltase gene and maltose transporter gene and use thereof, in particular, a brewer's yeast with high maltose assimilation ability, alcoholic beverages produced with said yeast, and a method for producing said beverages. More particularly, the present invention relates to a yeast, whose maltose assimilation is enhanced by amplifying expression level of MALR gene encoding MalRp, a maltase and maltose transporter transcription factor in brewer's yeast, especially non-ScMALR gene specific to a lager brewing yeast and to a method for producing alcoholic beverages with said yeast, etc.

### TECHNICAL FIELD

The present invention relates to a gene encoding transcriptional inducer for maltase gene and maltose transporter gene, and use thereof, in particular, brewer's yeast with superior maltose fermentability, alcoholic beverages produced with said yeast, and a method for producing said beverages. More particularly, the present invention relates to a yeast whose maltose assimilation ability is enhanced by amplifying expression level of MALR gene encoding a protein MalRp (transcriptional inducer for maltase gene and maltose transporter gene in brewer's yeast), especially non-ScMALR gene specific to a lager brewing yeast and to a method for producing alcoholic beverages with said yeast, etc.

### BACKGROUND ART

In beer production, while wort having about 11% extract concentration is fermented to obtain beer having about 4.5-5% alcohol concentration, high gravity brewing is sometimes adopted for improvement of beer productivity. The high gravity brewing is a method for producing beer with desired alcohol concentration, by fermenting wort with higher concentration than conventional wort, followed by diluting the resultant product with water. More specifically, the following measures are considered. (1) A higher temperature than conventional fermentation is adopted; (2) Airflow to wort is increased; (3) Yeast pitching rate is increased; and any combination of these measures. It is said that about 15% original wort extract concentration is maximum in high gravity brewing for conventional beer production.

A first problem in high gravity brewing with higher than 15% extract concentration is remarkable decrease of fermentation speed occurring at the middle to late stage of the fermentation. Main carbohydrates included in wort are maltose, maltotriose, glucose, fructose and sucrose. A yeast assimilates glucose, fructose and sucrose initially, then assimilates maltose and maltotriose. Accordingly only maltose and maltotriose exist in fermentation broth, during the middle to late stage of the fermentation. Moreover, there is overwhelmingly a lot of maltose with ratio of 3:1.

Maltose is transported into a yeast cell by maltose transporter, hydrolyzed to two glucoses by maltase, followed by conversion to carbon dioxide and ethanol mediated by Embden-Meyerhof pathway. These two enzymes are induced in the presence of maltose, but inhibited in the presence of glucose, at transcriptional level. It is known that transcription factor MalR plays an important role in transcriptional induction of maltase gene and maltose transporter gene in the presence of maltose, and transcription of MalR is also inhibited in the presence of glucose (Mol Cell Biol. 7:2477-2483, 1987, Curr Genet. 28:258-266, 1995).

Since about 17% of assimilable carbohydrates in wort is glucose, maltose metabolic genes of yeast are inhibited at the early stage of fermentation and causes a significant delay of maltose assimilation. This phenomena becomes more serious in high gravity brewing, which causes not only delay of fermentation but also remaining large amount of maltose as a residue of sugar at the completion of fermentation. Because of these problems, high gravity brewing (for example, fermentation with double concentration of regular wort) cannot be conducted sufficiently with use of the conventional techniques.

Japanese Patent Application Laid-open H1-153082 describes usage of baker's yeast transfected with a plasmid comprising a promoter for alcohol dehydrogenase gene that cannot be inhibited by glucose, maltase gene and maltose transporter gene for improvement of Dough fermentation by baker's yeast. Meanwhile, it is reported that a maltose transporter gene MAL6T of Saccharomyces cerevisiae was highly expressed in brewer's yeast, and high gravity brewing was achieved (Japanese Patent Application Laid-open No. H06-245750).

### DISCLOSURE OF INVENTION

Under the above situations, it is desired to provide a yeast that allows for a high gravity brewing without imparing fermentation speed and product quality.

The present inventors made extensive studies to solve the above problems and as a result, succeeded in identifying and isolating a gene encoding a transcriptional inducer for maltase gene and maltose transporter gene from beer yeast. Moreover, the present inventors produced transformed yeast in which the obtained gene was expressed to verify that maltose assimilation ability can be actually improved, thereby completing the present invention.

Thus, the present invention relates to a gene encoding a transcriptional inducer for maltase gene and maltose transporter gene existing in brewer's yeast, to a protein encoded by said gene, to a transformed yeast in which the expression of said gene is controlled, to a method for producing alcoholic beverages by using said transformed yeast in which the expression of said gene is controlled, and the like. More specifically, the present invention provides the following polynucleotides, a vector comprising said polynucleotide, a transformed yeast introduced with said vector, a method for producing alcoholic beverages by using said transformed yeast, and the like.

(1) A polynucleotide selected from the group consisting of:

(a) a polynucleotide comprising a polynucleotide consisting of the nucleotide sequence of SEQ ID NO: 1;

(b) a polynucleotide comprising a polynucleotide encoding a protein consisting of the amino acid sequence of SEQ ID NO: 2;

(c) a polynucleotide comprising a polynucleotide encoding a protein consisting of the amino acid sequence of SEQ ID NO: 2 in which one or more amino acids thereof are deleted, substituted, inserted and/or added, and having a transcriptional induction activity of maltase and maltose transporter gene;

(d) a polynucleotide comprising a polynucleotide encoding a protein having an amino acid sequence having 60% or higher identity with the amino acid sequence of SEQ ID NO: 2, and said protein having a transcriptional induction activity of maltase and maltose transporter gene;

(e) a polynucleotide comprising a polynucleotide which hybridizes to a polynucleotide consisting of a nucleotide sequence complementary to the nucleotide sequence of SEQ ID NO: 1 under stringent conditions and which encodes a protein having a transcriptional induction activity of maltase and maltose transporter gene; and

(f) a polynucleotide comprising a polynucleotide which hybridizes to a polynucleotide consisting of a nucleotide sequence complementary to the nucleotide sequence of the polynucleotide encoding the protein having the amino acid sequence of SEQ ID NO: 2 under stringent conditions, and which encodes a protein having a transcriptional induction activity of maltase and maltose transporter gene.

(2) The polynucleotide according to (1) above selected from the group consisting of:

(g) a polynucleotide comprising a polynucleotide encoding a protein consisting of the amino acid sequence of SEQ ID NO: 2, or encoding the amino acid sequence of SEQ ID NO: 2 in which 1 to 10 amino acids thereof are deleted, substituted, inserted, and/or added, and wherein said protein has a transcriptional induction activity of maltase and maltose transporter gene;

(h) a polynucleotide comprising a polynucleotide encoding a protein having 90% or higher identity with the amino acid sequence of SEQ ID NO: 2, and having a transcriptional induction activity of maltase and maltose transporter gene; and

(i) a polynucleotide comprising a polynucleotide which hybridizes to a polynucleotide consisting of a nucleotide sequence of SEQ ID NO: 1 or which hybridizes to a polynucleotide consisting of a nucleotide sequence complementary to the nucleotide sequence of SEQ ID NO: 1, under high stringent conditions, which encodes a protein having a transcriptional induction activity of maltase and maltose transporter gene.

(3) The polynucleotide according to (1) above comprising a polynucleotide consisting of the nucleotide sequence of SEQ ID NO: 1.

(4) The polynucleotide according to (1) above comprising a polynucleotide encoding a protein consisting of the amino acid sequence of SEQ ID NO: 2.

(5) The polynucleotide according to any one of (1) to (4) above, wherein the polynucleotide is DNA.

(6) A protein encoded by the polynucleotide according to any one of (1) to (5) above.

(7) A vector containing the polynucleotide according to any one of (1) to (5) above.

(7a) The vector of (7) above, which comprises the expression cassette comprising the following components:

(x) a promoter that can be transcribed in a yeast cell;

(y) any of the polynucleotides described in (1) to (5) above linked to the promoter in a sense or antisense direction; and

(z) a signal that can function in a yeast with respect to transcription termination and polyadenylation of a RNA molecule.

(7b) The vector of (7) above, which comprises the expression cassette comprising the following components:

(x) a promoter that can be transcribed in a yeast cell;

(y) any of the polynucleotides described in (1) to (5) above linked to the promoter in a sense direction; and

(z) a signal that can function in a yeast with respect to transcription termination and polyadenylation of a RNA molecule.

(8) A yeast into which the vector according to any one of (7) to (7b) above has been introduced.

(9) The yeast according to (8) above, wherein maltose assimilation ability is increased by introducing the vector according to any one of (7) to (7b) above.

(10) The yeast according to (9) above, wherein maltose assimilation ability is increased by increasing an expression level of the protein of (6) above.

(11) A method for producing an alcoholic beverage by using the yeast according to any one of Claims (8) to (10) above.

(12) The method according to (11) above, wherein the brewed alcoholic beverage is a malt beverage.

(13) An alcoholic beverage produced by the method according to (11) or (12) above.

(14) A method for assessing a test yeast for its maltose assimilation ability, comprising using a primer or probe designed based on the nucleotide sequence of a gene having the nucleotide sequence of SEQ ID NO: 1 and encoding a transcriptional inducer for maltase gene and maltose transporter gene.

(14a) A method for selecting a yeast having increased maltose assimilation ability by using the method described in (14) above.

(14b) A method for producing an alcoholic beverage (for example, beer) by using the yeast selected with the method described in (14a) above.

(15) A method for assessing a test yeast for its high maltose assimilation ability, comprising: culturing the test yeast; and measuring the expression level of the gene having the nucleotide sequence of SEQ ID NO: 1 and encoding a transcriptional inducer for maltase gene and maltose transporter gene.

(15a) A method for selecting a yeast having superior maltose assimilation ability, which comprises assessing a test yeast by the method described in (15) above and selecting a yeast having a high expression level of gene encoding a transcriptional inducer for maltase gene and maltose transporter gene.

(15b) A method for producing an alcoholic beverage (for example, beer) by using the yeast selected with the method described in (15a) above.

(16) A method for selecting a yeast, comprising: culturing test yeasts; quantifying the protein of (6) above or measuring the expression level of the gene having the nucleotide sequence of SEQ ID NO: 1 and encoding a transcriptional inducer for maltase gene and maltose transporter gene; and selecting a test yeast having an amount of the protein or the gene expression level according to desired maltose assimilation ability.

(17) The method for selecting a yeast according to (16) above, comprising: culturing a reference yeast and test yeasts; measuring for each yeast the expression level of the gene having the nucleotide sequence of SEQ ID NO: 1 and encoding a transcriptional inducer for maltase gene and maltose transporter gene; and selecting a test yeast having gene expression level higher than that in the reference yeast.

(18) The method for selecting a yeast according to (16) above, comprising: culturing a reference yeast and test yeasts; quantifying the protein according to (6) above in each yeast; and selecting a test yeast having a larger amount of the protein than that in the reference yeast.

(19) A method for producing an alcoholic beverage comprising: conducting fermentation using the yeast according to any one of (8) to (10) above or a yeast selected by the method according to any one of (16) to (18) above.

According to the method for producing alcoholic beverages using transformed yeast of the present invention, assimilation of maltose is not inhibited even in the presence of glucose. As a result, a beer brewing with high wort concentration can be achieved since fermentation speed is increased due to maltose assimilation prior to disappearance of glucose.

### BRIEF DESCRIPTION OF DRAWINGS

FIG. 1 shows the cell growth with time upon beer fermentation test. The horizontal axis represents fermentation time while the vertical axis represents optical density at 660 mn (OD660).

FIG. 2 shows the extract (sugar) consumption with time upon beer fermentation test. The horizontal axis represents fermentation time while the vertical axis represents apparent extract concentration(w/w/%).

FIG. 3 shows the expression profile of non-ScMALR gene in yeasts upon beer fermentation test. The horizontal axis represents fermentation time while the vertical axis represents the intensity of detected signal.

### BEST MODES FOR CARRYING OUT TE INVENTION

The present inventors conceived that maltose could be assimilated more efficiently by increasing transcriptional induction activity of maltase and maltose transporter gene. The present inventors made extensive studies based on the conception, isolated and identified non-ScMALR gene encoding a transcriptional inducer for maltase gene and maltose transporter gene which is specific to lager brewing yeast, based on the lager brewing yeast genome information mapped according to the method disclosed in Japanese Patent Application Laid-Open No. 2004-283169. The nucleotide sequence of the gene is represented by SEQ ID NO: 1. Further, an amino acid sequence of a protein encoded by the gene is represented by SEQ ID NO: 2.

### 1. Polynucleotide of the Invention

First of all, the present invention provides (a) a polynucleotide comprising a polynucleotide of the nucleotide sequence of SEQ ID NO: 1; and (b) a polynucleotide comprising a polynucleotide encoding a protein of the amino acid sequence of SEQ ID NO: 2. The polynucleotide can be DNA or RNA.

The target polynucleotide of the present invention is not limited to the polynucleotide encoding a transcriptional inducer for maltase gene and maltose transporter gene described above, and may include other polynucleotides encoding proteins having equivalent functions to said protein. Proteins with equivalent functions include, for example, (c) a protein of an amino acid sequence of SEQ ID NO: 2 with one or more amino acids thereof being deleted, substituted, inserted and/or added and having a transcriptional induction activity of maltase and maltose transporter gene.

Such proteins include a protein consisting of an amino acid sequence of SEQ ID NO: 2 with, for example, 1 to 100, 1 to 90, 1 to 80, 1 to 70, 1 to 60, 1 to 50, 1 to 40, 1 to 39, 1 to 38, 1 to 37, 1 to 36, 1 to 35, 1 to 34, 1 to 33, 1 to 32, 1 to 31, 1 to 30, 1 to 29, 1 to 28, 1 to 27, 1 to 26, 1 to 25, 1 to 24, 1 to 23, 1 to 22, 1 to 21, 1 to 20, 1 to 19; 1 to 18, 1 to 17, 1 to 16, 1 to 15, 1 to 14, 1 to 13, 1 to 12, 1 to 11, 1 to 10, 1 to 9, 1 to 8, 1 to 7, 1 to 6 (1 to several amino acids), 1 to 5, 1 to 4, 1 to 3, 1 to 2, or 1 amino acid residues thereof being deleted, substituted, inserted and/or added and having a transcriptional induction activity of maltase and maltose transporter gene. In general, the number of deletions, substitutions, insertions, and/or additions is preferably smaller. In addition, such proteins include (d) a protein having an amino acid sequence with about 60% or higher, about 70% or higher, 71% or higher, 72% or higher, 73% or higher, 74% or higher, 75% or higher, 76% or higher, 77% or higher, 78% or higher, 79% or higher, 80% or higher, 81% or higher, 82% or higher, 83% or higher, 84% or higher, 85% or higher, 86% or higher, 87% or higher, 88% or higher, 89% or higher, 90% or higher, 91% or higher, 92% or higher, 93% or higher, 94% or higher, 95% or higher, 96% or higher, 97% or higher, 98% or higher, 99% or higher, 99.1% or higher, 99.2% or higher, 99.3% or higher, 99.4% or higher, 99.5% or higher, 99.6% or higher, 99.7% or higher, 99.8% or higher, or 99.9% or higher identity with the amino acid sequence of SEQ ID NO: 2, and having a transcriptional induction activity of maltase and maltose transporter gene. In general, the percentage identity is preferably higher.

In addition, transcriptional induction activity of maltase and maltose transporter gene may be measured, by quantification of transcript level of the gene (mRNA). mRNA may be quantified, by Northern hybridization or quantitative RT-PCR (CURRENT PROTOCOLS IN MOLECULAR BIOLOGY, John Wiley & Sons 1994-2003).

Furthermore, the present invention also contemplates (e) a polynucleotide comprising a polynucleotide which hybridizes to a polynucleotide consisting of a nucleotide sequence complementary to the nucleotide sequence of SEQ ID NO: 1 under stringent conditions and which encodes a protein having a transcriptional induction activity of maltase and maltose transporter gene and (f) a polynucleotide comprising a polynucleotide which hybridizes to a polynucleotide complementary to a nucleotide sequence of encoding a protein of SEQ ID NO: 2 under stringent conditions, and which encodes a protein having a transcriptional induction activity of maltase and maltose transporter gene.

Herein, “a polynucleotide that hybridizes under stringent conditions” refers to nucleotide sequence, such as a DNA, obtained by a colony hybridization technique, a plaque hybridization technique, a southern hybridization technique or the like using all or part of polynucleotide of a nucleotide sequence complementary to the nucleotide sequence of SEQ ID NO: 1 or polynucleotide encoding the amino acid sequence of SEQ ID NO: 2 as a probe. The hybridization method may be a method described, for example, in MOLECULAR CLONING 3rd Ed., CURRENT PROTOCOLS IN MOLECULAR BIOLOGY, John Wiley & Sons 1987-1997, and so on.

The term “stringent conditions” as used herein may be any of low stringency conditions, moderate stringency conditions or high stringency conditions. “Low stringency conditions” are, for example, 5×SSC, 5×Denhardt's solution, 0.5% SDS, 50% formamide at 32° C. “Moderate stringency conditions” are, for example, 5×SSC, 5×Denhardt's solution, 0.5% SDS, 50% formamide at 42° C. “High stringency conditions” are, for example, 5×SSC, 5×Denhardt's solution, 0.5% SDS, 50% formamide at 50° C. Under these conditions, a polynucleotide, such as a DNA, with higher homology is expected to be obtained efficiently at higher temperature, although multiple factors are involved in hybridization stringency including temperature, probe concentration, probe length, ionic strength, time, salt concentration and others, and one skilled in the art may appropriately select these factors to realize similar stringency.

When a commercially available kit is used for hybridization, for example, Alkphos Direct Labeling Reagents (Amersham Pharmacia) may be used. In this case, according to the attached protocol, after incubation with a labeled probe overnight, the membrane is washed with a primary wash buffer containing 0.1% (w/v) SDS at 55° C., thereby detecting hybridized polynucleotide, such as DNA.

Other polynucleotides that can be hybridized include polynucleotides having about 60% or higher, about 70% or higher, 71% or higher, 72% or higher, 73% or higher, 74% or higher, 75% or higher, 76% or higher, 77% or higher, 78% or higher, 79% or higher, 80% or higher, 81% or higher, 82% or higher, 83% or higher, 84% or higher, 85% or higher, 86% or higher, 87% or higher, 88% or higher, 89% or higher, 90% or higher, 91% or higher, 92% or higher, 93% or higher, 94% or higher, 95% or higher, 96% or higher, 97% or higher, 98% or higher, 99% or higher, 99.1% or higher, 99.2% or higher, 99.3% or higher, 99.4% or higher, 99.5% or higher, 99.6% or higher, 99.7% or higher, 99.8% or higher or 99.9% or higher identity to polynucleotide encoding the amino acid sequence of SEQ ID NO: 2 as calculated by homology search software, such as FASTA and BLAST using default parameters.

Identity between amino acid sequences or nucleotide sequences may be determined using algorithm BLAST by Karlin and Altschul (Proc. Natl. Acad. Sci. USA, 87: 2264-2268, 1990; Proc. Natl. Acad Sci. USA, 90: 5873, 1993). Programs called BLASTN and BLASTX based on BLAST algorithm have been developed (Altschul SF et al., J. Mol. Biol 215: 403, 1990). When a nucleotide sequence is sequenced using BLASTN, the parameters are, for example, score=100 and word length=12. When an amino acid sequence is sequenced using BLASTX, the parameters are, for example, score=50 and word length=3. When BLAST and Gapped BLAST programs are used, default parameters for each of the programs are employed.

### 2. Protein of the Present Invention

The present invention also provides proteins encoded by any of the polynucleotides (a) to (i) above. A preferred protein of the present invention comprises an amino acid sequence of SEQ ID NO: 2 with one or several amino acids thereof being deleted, substituted, inserted and/or added, and having a transcriptional induction activity of maltase and maltose transporter gene.

Such protein includes those having an amino acid sequence of SEQ ID NO: 2 with amino acid residues thereof of the number mentioned above being deleted, substituted, inserted and/or added and having a transcriptional induction activity of maltase and maltose transporter gene. In addition, such protein includes those having homology as described above with the amino acid sequence of SEQ ID NO: 2 and having a transcriptional induction activity of maltase and maltose transporter gene.

Such proteins may be obtained by employing site-directed mutation described, for example, in MOLECULAR CLONING 3rd Ed., CURRENT PROTOCOLS IN MOLECULAR BIOLOGY, Nuc. Acids. Res., 10: 6487 (1982), Proc. Natl. Acad Sci. USA 79: 6409 (1982), Gene 34: 315 (1985), Nuc. Acids. Res., 13: 4431 (1985), Proc. Natl. Acad. Sci. USA 82: 488 (1985).

Deletion, substitution, insertion and/or addition of one or more amino acid residues in an amino acid sequence of the protein of the invention means that one or more amino acid residues are deleted, substituted, inserted and/or added at any one or more positions in the same amino acid sequence. Two or more types of deletion, substitution, insertion and/or addition may occur concurrently.

Hereinafter, examples of mutually substitutable amino acid residues are enumerated. Amino acid residues in the same group are mutually substitutable. The groups are provided below.

Group A: leucine, isoleucine, norleucine, valine, norvaline, alanine, 2-aminobutanoic acid, methionine, o-methylserine, t-butylglycine, t-butylalanine, cyclohexylalanine; Group B: asparatic acid, glutarnic acid, isoasparatic acid, isoglutamic acid, 2-aminoadipic acid, 2-aminosuberic acid; Group C: asparagine, glutamine; Group D: lysine, arginine, ornithine, 2,4-diaminobutanoic acid, 2,3-diaminopropionic acid; Group E: proline, 3-hydroxyproline, 4-hydroxyproline; Group F: serine, threonine, homoserine; and Group G: phenylalanine, tyrosine.

The protein of the present invention may also be produced by chemical synthesis methods such as Fmoc method (fluorenyhnethyloxycarbonyl method) and tBoc method (t-butyloxycarbonyl method). In addition, peptide synthesizers available from, for example, Advanced ChemTech, PerkinElmer, Pharmacia, Protein Technology Instrument, Synthecell-Vega, PerSeptive, Shimadzu Corp. can also be used for chemical synthesis.

3. Vector of the Invention and Yeast Transformed with the Vector

The present invention then provides a vector comprising the polynucleotide described above. The vector of the present invention is directed to a vector including any of the polynucleotides described in (a) to (i) above. Generally, the vector of the present invention comprises an expression cassette including as components (x) a promoter that can transcribe in a yeast cell; (y) a polynucleotide described in any of (a) to (i) above that is linked to the promoter in sense or antisense direction; and (z) a signal that functions in the yeast with respect to transcription termination and polyadenylation of RNA molecule.

A vector introduced in the yeast may be any of a multicopy type (YEp type), a single copy type (YCp type), or a chromosome integration type (YIp type). For example, YEp24 (J. R. Broach et al., EXPERIMENTAL MANIPULATION OF GENE EXPRESSION, Academic Press, New York, 83, 1983) is known as a YEp type vector, YCp50 (M. D. Rose et al., Gene 60: 237, 1987) is known as a YCp type vector, and YIp5 (K. Struhl et al., Proc. Natl. Acad Sci. USA, 76: 1035, 1979) is known as a YIp type vector, all of which are readily available.

Promoters/terminators for adjusting gene expression in yeast may be in any combination as long as they function in the yeast for practical use and they are not influenced by sugar or amino acids in fermentation broth. For example, a promoter of glyceraldehydes 3-phosphate dehydrogenase gene (TDH3), or a promoter of 3-phosphoglycerate kinase gene (PGK1) may be used. These genes have previously been cloned, described in detail, for example, in M. F. Tuite et al., EMBO J., 1, 603 (1982), and are readily available by known methods.

Since an auxotrophy marker cannot be used as a selective marker upon transformation for a yeast for practical use, for example, a geneticin-resistant gene (G418r), a copper-resistant gene (CUP1) (Marin et al., Proc. Natl. Acad. Sci. USA, 81, 337 1984) or a cerulenin-resistant gene (fas2m, PDR4) (Junji Inokoshi et al., Biochemistry, 64, 660, 1992; and Hussain et al., Gene, 101: 149, 1991, respectively) may be used.

A vector constructed as described above is introduced into a host yeast. Examples of the host yeast include any yeast that can be used for brewing, for example, brewer's yeasts for beer, wine and sake. Specifically, yeasts such as genus Saccharomyces may be used. According to the present invention, a lager brewing yeast, for example, Saccharomyces pastorianus W34/70, etc., Saccharomyces carlsbergensis NCYC453 or NCYC456, etc., or Saccharomyces cerevisiae NBRC1951, NBRC1952, NBRC1953 or NBRC1954, etc., may be used. In addition, whisky yeasts such as Saccharomyces cerevisiae NCYC90, wine yeasts such as wine yeasts #1, 3 and 4 from the Brewing Society of Japan, and sake yeasts such as sake yeast #7 and 9 from the Brewing Society of Japan, may also be used but not limited thereto. In the present invention, lager brewing yeasts such as Saccharomyces pastorianus may be used preferably.

A yeast transformation method may be a generally used known method. For example, methods that can be used include but not limited to an electroporation method (Meth. Enzym., 194: 182 (1990)), a spheroplast method (Proc. Natl. Acad. Sci. USA, 75: 1929(1978)), a lithium acetate method (J. Bacteriology, 153: 163 (1983)), and methods described in Proc. Natl. Acad. Sci. USA, 75: 1929 (1978), METHODS IN YEAST GENETICS, 2000 Edition: A Cold Spring Harbor Laboratory Course Manual.

More specifically, a host yeast is cultured in a standard yeast nutrition medium (e.g., YEPD medium (Genetic Engineering. Vol. 1, Plenum Press, New York, 117(1979)), etc.) suchx that OD600 nm will be 1 to 6. This culture yeast is collected by centrifugation, washed and pre-treated with alkali metal ion, preferably lithium ion at a concentration of about 1 to 2 M. After the cell is left to stand at about 30° C. for about 60 minutes, it is left to stand with DNA to be introduced (about 1 to 20 μg) at about 30° C. for about another 60 minutes. Polyethyleneglycol, preferably about 4,000 Dalton of polyethyleneglycol is added to a final concentration of about 20% to 50%. After leaving at about 30° C. for about 30 minutes, the cell is heated at about 42° C. for about 5 minutes. Preferably, this cell suspension is washed with a standard yeast nutrition medium, added to a predetermined amount of fresh standard yeast nutrition medium and left to stand at about 30° C. for about 60 minutes. Thereafter, it is seeded to a standard agar medium containing an antibiotic or the like as a selective marker to obtain a transformant.

Other general cloning techniques may be found, for example, in MOLECULAR CLONING 3rd Ed., and METHODS IN YEAST GENETICS, A LABORATORY MANUAL (Cold Spring Harbor Laboratory Press, Cold Spring Harbor, N.Y.).

### 4. Method of Producing Alcoholic Beverages According to the Present Invention and Alcoholic Beverages Produced by the Method

Alcoholic beverages can be produced with use of high wort concentration for a shorter period of time by introducing the above-mentioned vector of the present invention to a yeast suitable for brewring of alcoholic beverage to be produced, and using the yeast. Furthermore, a yeast having superior maltose assimilation ability can be obtained by selecting yeast by the yeast assessment method of the present invention described below. The target alcoholic beverages include, for example, but not limited to beer, beer-taste beverages such as sparkling liquor (happoushu) and the like.

In order to produce these products, a known technique can be used except that a brewer's yeast obtained according to the present invention is used in the place of a parent strain. Since starting materials, manufacturing equipment, manufacturing control and the like may be the same as the conventional ones; it can be performed without increasing cost.

### 5. Yeast Assessment Method of the Invention

The present invention relates to a method for assessing a test yeast for its maltose assimilation ability by using a primer or a probe designed based on a nucleotide sequence of a gene having the nucleotide sequence of SEQ ID NO: 1 and encoding a transcriptional inducer for maltase gene and maltose transporter gene. General technique for such assessment method is known and is described in, for example, WO01/040514, Japanese Laid-Open Patent Application No. H8-205900 or the like. This assessment method is described in below.

First genome of a test yeast is prepared. For this preparation, any known method such as Hereford method or potassium acetate method may be used (e.g., METHODS IN YEAST GENETICS, Cold Spring Harbor Laboratory Press, 130 (1990)). Using a primer or a probe designed based on a nucleotide sequence (preferably, ORF sequence) of the gene encoding a transcriptional inducer for maltase gene and maltose transporter gene, the existence of the gene or a sequence specific to the gene is determined in the test yeast genome obtained. The primer or the probe may be designed according to a known technique.

Detection of the gene or the specific sequence may be carried out by employing a known technique. For example, a polynucleotide including part or all of the specific sequence or a polynucleotide including a nucleotide sequence complementary to said nucleotide sequence is used as one primer, while a polynucleotide including part or all of the sequence upstream or downstream from this sequence or a polynucleotide including a nucleotide sequence complementary to said nucleotide sequence, is used as another primer to amplify a nucleic acid of the yeast by a PCR method, thereby determining the existence of amplified products and molecular weight of the amplified products. The number of bases of polynucleotide used for a primer is generally 10 base pairs (bp) or more, and preferably 15 to 25 bp. In general, the number of bases between the primers is suitably 300 to 2000 bp.

The reaction conditions for PCR are not particularly limited but may be, for example, a denaturation temperature of 90 to 95° C., an annealing temperature of 40 to 60° C., an elongation temperature of 60 to 75° C., and the number of cycle of 10 or more. The resulting reaction product may be separated, for example, by electrophoresis using agarose gel to determine the molecular weight of the amplified product. This method allows prediction and assessment of maltose assimliation ability of yeast as determined by whether the molecular weight of the amplified product is a size that contains the DNA molecule of the specific part. In addition, by analyzing the nucleotide sequence of the amplified product, the property may be predicted and/or assessed more precisely.

Moreover, in the present invention, a test yeast is cultured to measure an expression level of the gene encoding a transcriptional inducer for maltase gene and maltose transporter gene and having the nucleotide sequence of SEQ ID NO: 1 to assess the test yeast for its maltose assimilation ability. Measurement of expression level of the gene encoding a transcriptional inducer for maltase gene and maltose transporter gene can be performed by culturing test yeast and then quantifying MRNA or a protein resulting from the gene. The quantification of mRNA or protein may be carried out by employing a known technique. For example, mRNA may be quantified, by Northern hybridization or quantitative RT-PCR, while protein may be quantified, for example, by Western blotting (CURRENT PROTOCOLS IN MOLECULAR BIOLOGY, John Wiley & Sons 1994-2003). In addition, expression level of the gene in the test yeast can be estimated by measuring maltose level in a fermentation broth obtained at fermentation of the test yeast.

Furthermore, test yeasts are cultured and expression levels of the gene encoding a transcriptional inducer for maltase gene and maltose transporter gene having the nucleotide sequence of SEQ ID NO: 1 are measured to select a test yeast with the gene expression level according to the target maltose assimilation ability, thereby a yeast favorable for brewing desired alcoholic beverages can be selected. In addition, a reference yeast and a test yeast may be cultured so as to measure and compare the expression level of the gene in each of the yeasts, thereby a favorable test yeast can be selected. More specifically, for example, a reference yeast and one or more test yeasts are cultured and an expression level of the gene encoding a transcriptional inducer for maltase gene and maltose transporter gene having the nucleotide sequence of SEQ ID NO: 1 is measured in each yeast. By selecting a test yeast with the gene expressed higher than that in the reference yeast, a yeast suitable for brewing desired alcoholic beverages or production of useful materials can be selected.

Alternatively, test yeasts are cultured and a yeast with maltose assimilation ability is selected, thereby a yeast suitable for brewing desired alcoholic beverages or production of useful materials can be selected.

In these cases, the test yeasts or the reference yeast may be, for example, a yeast introduced with the vector of the invention, an artificially mutated yeast or a naturally mutated yeast. The mutation treatment may employ any methods including, for example, physical methods such as ultraviolet irradiation and radiation irradiation, and chemical methods associated with treatments with drugs such as EMS (ethylmethane sulphonate) and N-methyl-N-nitrosoguanidine (see, e.g, Yasuji Oshima Ed., BIOCHEMISTRY EXPERIMENTS vol. 39, Yeast Molecular Genetic Experiments, pp. 67-75, JSSP).

In addition, examples of yeasts used as the reference yeast or the test yeasts include any yeasts, for example, brewer's yeasts for beer, wine, sake and the like. More specifically, yeasts such as genus Saccharomyces may be used (e.g., Saccharomyces pastorianus, Saccharomyces cerevisiae, and Saccharomyces carlsbergensis). According to the present invention, a lager brewing yeast, for example, Saccharomyces pastorianus W34/70; Saccharomyces carlsbergensis NCYC453 or NCYC456; or Saccharomyces cerevisiae NBRC1951, NBRC1952, NBRC1953 or NBRC1954, etc., may be used. Further, wine yeasts such as wine yeasts #1, 3 and 4 from the Brewing Society of Japan; and sake yeasts such as sake yeast #7 and 9 from the Brewing Society of Japan, may also be used but not limited thereto. In the present invention, lager brewing yeasts such as Saccharomyces pastorianus may preferably be used. The reference yeast and the test yeasts may be selected from the above yeasts in any combination.

### EXAMPLES

Hereinafter, the present invention will be described in more detail with reference to working examples. The present invention, however, is not limited to the examples described below.

### Example 1

### Cloning of Gene Encoding Transcriptional Inducer for Maltase Gene and Maltose Transporter Gene (non-ScMALR)

A gene encoding a transcriptional inducer for maltase gene and maltose transporter gene of lager brewing yeast (non-ScMALR) (SEQ ID NO: 1) was found as a result of a search utilizing the comparison database described in Japanese Patent Application Laid-Open No. 2004-283169. Based on the acquired nucleotide sequence information, primers non-ScMALR_F (SEQ ID NO: 3) and non-ScMALR R (SEQ ID NO: 4) were designed to amplify the full-length of the gene. PCR was carried out using chromosomal DNA of a genome sequencing strain, Saccharomyces pastorianus Weihenstephan 34/70 (sometimes abbreviated as “W34/70 strain”), as a template to obtain DNA fragments including the full-length gene of non-ScMALR.

The non-ScMALR gene fragments thus obtained were inserted into pCR2.1-TOPO vector (Invitrogen) by TA cloning. The nucleotide sequences of the non-ScMALR gene were analyzed by Sanger's method (F. Sanger, Science, 214: 1215, 1981) to confirm the nucleotide sequence.

### Example 2

### Analysis of Expression of non-ScMALR Gene During Beer Fermentation

A beer fermentation test was conducted using a lager brewing yeast, Saccharomyces pastorianus W34/70, and mRNA extracted from the lager brewing yeast during fermentation was detected by a beer yeast DNA microarray.

| | |
|---|---|
| Wort extract concentration | 12.69% |
| Wort content | 70 L |
| Wort dissolved oxygen concentration | 8.6 ppm |
| Fermentation temperature | 15° C. |
| Yeast pitching rate | 12.8 × 10⁶ cells/mL |

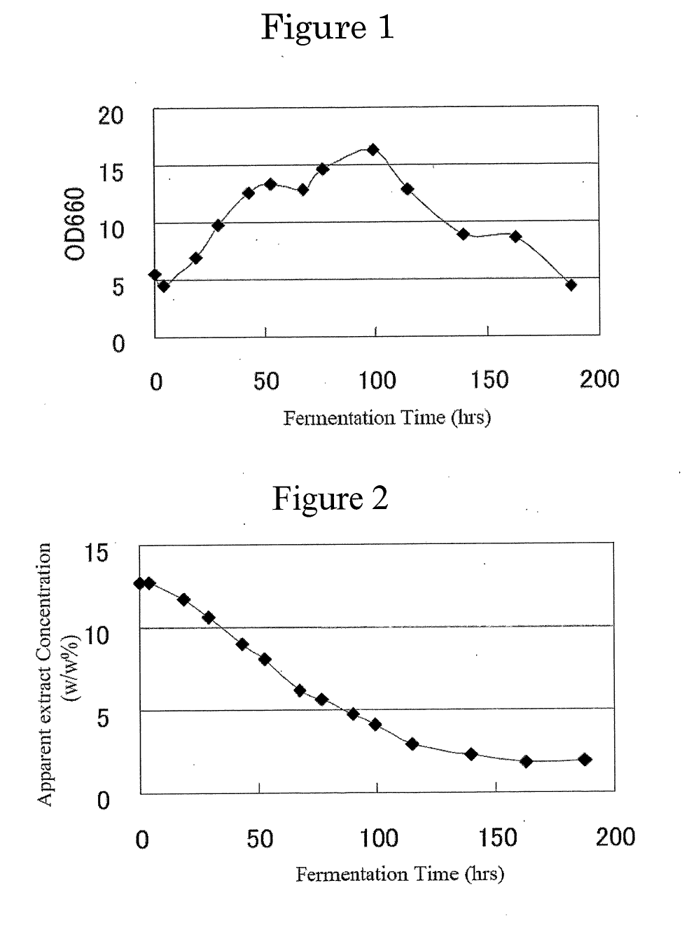

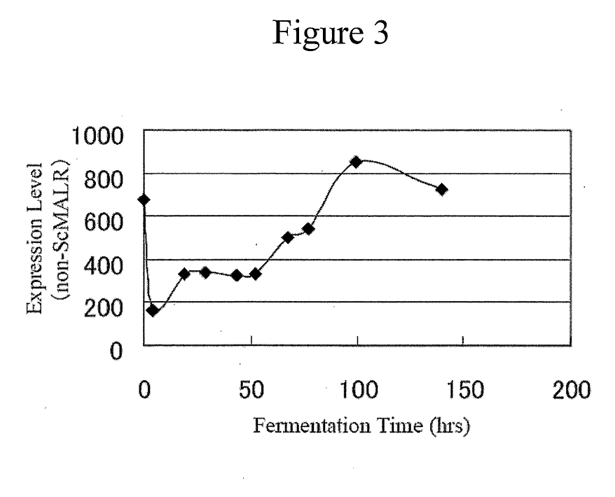

The fermentation liquor was sampled over time, and the time-course changes in amount of yeast cell growth (FIG. 1) and apparent extract concentration (FIG. 2) were observed. Simultaneously, yeast cells were sampled to prepare mRNA, and the prepared mRNA was labeled with biotin and was hybridized to a beer yeast DNA microarray. The signal was detected using GeneChip Operating system (GCOS; GeneChip Operating Software 1.0, manufactured by Affymetrix Co). Expression pattern of the non-ScMALR gene is shown in FIG. 3. This result confirmed the expression of the non-ScMALR gene in the general beer fermentation.

### Example 3

### Construction of non-ScMALR Highly Expressed Strain

The non-ScMALR/pCR2.1-TOPO described in Example 1 was digested with the restriction enzymes SacI and NotI to prepare a DNA fragment containing the entire length of the protein-encoding region. This fragment was ligated to pYCGPYNot treated with the restriction enzymes SacI and NotI, thereby constructing the non-ScMALR high expression vector non-ScMALR/pYCGPYNot. pYCGPYNot is a YCp-type yeast expression vector. A gene inserted is highly expressed by the pyruvate kinase gene PYK1 promoter. The geneticin-resistant gene G418^{r} is included as the selectable marker in the yeast, and the ampicillin-resistant gene Amp^{r} as the selectable marker in Escherichia coli.

Using the high expression vector prepared by the above method, a Saccharomyces pastorianus UPMT3 strain was transformed by the method described in Japanese Patent Application Laid-open No. H07-303475. UPMT3 is a strain in which maltose transporter gene, MAL6T (as described in Japanese Patent Application Laid-open No. H06-245750) is introduced into chromosome of Saccharomyces pastorianus BH84 strain with use of YIp type high expression plasmid, pUP3GLP (as described in Japanese Patent Application Laid-open No. 2000-316559). The transformants were selected on a YPD plate medium (1% yeast extract, 2% polypeptone, 2% glucose and 2% agar) containing 300 mg/L of geneticin.

### Example 4

### Beer Fermentation with High Wort Concentration

A fermentation test for the parent strain and non-ScMALR highly expressed strain obtained in Example 3 was carried out under the following conditions:

| | |
|---|---|
| Wort extract concentration | 16.9% (5% glucose is added to 12% wort) |
| Wort content | 20 ml |
| Fermentation temperature | 28° C. (constant) |

Yeast pitching rate was adjusted to make OD660=1.1 at the onset of fermentation. The concentration of an extract, glucose, maltose and maltotriose in the fermentation broth after 44.5 hours form the onset of fermentation was measured by liquid chromatography. As shown in Table 1, the extract concentration in the case of non-ScMALR highly expressed strain was lower than the parent strain, and fermentation degree was increased by 4.2% after 44.5 hours from the onset of fermentation.

Further, analysis of carbohydrates in the fermentation broth at the completion of the fermentation, shows acceleration of assimilation of maltose and maltotriose as compared with that of parent strain as shown in Table 2.

TABLE 1

| | Wort Extract | Fermentation |
|---|---|---|
| Strain | Concentration (%) | Degree (%) |
| Parent Strain | 3.04 | 82.0 |
| (UPMT3) | | |
| Non-ScMALR | 2.34 | 86.2 |
| Highly Expressed | | |
| Strain | | |
| | | (%) |

TABLE 2

| Strain | Glucose | Maltose | Maltotoriose |
|---|---|---|---|
| Parent Strain | 0.03 | 0.05 | 0.92 |
| (UPMT3) | | | |
| Non-ScMALR | 0.03 | 0.22 | 0.6 |
| Highly Expressed | | | |
| Strain | | | |
| | | | (%) |

### INDUSTRIAL APPLICABILITY

The method for producing alcoholic beverages of the present invention can make it possible to produce alcoholic beverages for a shorter period of time even in high gravity brewing since maltose assimilation ability is enhanced.

1. A polynucleotide selected from the group consisting of:
(a) a polynucleotide comprising a polynucleotide consisting of the nucleotide sequence of SEQ ID NO: 1;
(b) a polynucleotide comprising a polynucleotide encoding a protein consisting of the amino acid sequence of SEQ ID NO: 2;
(c) a polynucleotide comprising a polynucleotide encoding a protein consisting of the amino acid sequence of SEQ ID NO: 2 in which one or more amino acids thereof are deleted, substituted, inserted and/or added, and having a transcriptional induction activity of maltase and maltose transporter gene;
(d) a polynucleotide comprising a polynucleotide encoding a protein having an amino acid sequence having 60% or higher identity with the amino acid sequence of SEQ ID NO: 2, and said protein having a transcriptional induction activity of maltase and maltose transporter gene;
(e) a polynucleotide comprising a polynucleotide which hybridizes to a polynucleotide consisting of a nucleotide sequence complementary to the nucleotide sequence of SEQ ID NO: 1 under stringent conditions, and which encodes a protein having a transcriptional induction activity of maltase and maltose transporter gene; and
(f) a polynucleotide comprising a polynucleotide which hybridizes to a polynucleotide consisting of a nucleotide sequence complementary to the nucleotide sequence of the polynucleotide encoding the protein having the amino acid sequence of SEQ ID NO: 2 under stringent conditions, and which encodes a protein having a transcriptional induction activity of maltase and maltose transporter gene.

2. The polynucleotide according to claim 1 selected from the group consisting of:
(g) a polynucleotide comprising a polynucleotide encoding a protein consisting of the amino acid sequence of SEQ ID NO: 2, or encoding the amino acid sequence of SEQ ID NO: 2 in which 1 to 10 amino acids thereof are deleted, substituted, inserted, and/or added, and wherein said protein has a transcriptional induction activity of maltase and maltose transporter gene;
(h) a polynucleotide comprising a polynucleotide encoding a protein having 90% or higher identity with the amino acid sequence of SEQ ID NO: 2, and having a transcriptional induction activity of maltase and maltose transporter gene; and
(i) a polynucleotide comprising a polynucleotide which hybridizes to a polynucleotide consisting of a nucleotide sequence of SEQ ID NO: 1 or which hybridizes to a polynucleotide consisting of a nucleotide sequence complementary to the nucleotide sequence of SEQ ID NO: 1, under high stringent conditions, which encodes a protein having a transcriptional induction activity of maltase and maltose transporter gene.

3. The polynucleotide according to claim 1 comprising a polynucleotide consisting of the nucleotide sequence of SEQ ID NO: 1.

4. The polynucleotide according to claim 1 comprising a polynucleotide encoding a protein consisting of the amino acid sequence of SEQ ID NO: 2.

5. The polynucleotide according to claim 1, wherein the polynucleotide is DNA.

6. A protein encoded by the polynucleotide according to claim 1.

7. A vector containing the polynucleotide according to claim 1.

8. A yeast into which the vector according to claim 7 has been introduced.

9. The yeast according to claim 8, wherein maltose assimilation ability is increased by introducing the vector containing the polynucleotide.

10. The yeast according to claim 8, wherein maltose assimilation ability is increased by increasing an expression level of the protein encoded by the polynucleotide.

11. A method for producing an alcoholic beverage by using the yeast according to claim 8.

12. The method according to claim 11, wherein the brewed alcoholic beverage is a malt beverage.

13. An alcoholic beverage produced by the method according to claim 11.

14. A method for assessing a test yeast for its maltose assimilation ability, comprising using a primer or probe designed based on the nucleotide sequence of a gene having the nucleotide sequence of SEQ ID NO: 1 and encoding a transcriptional inducer for maltase gene and maltose transporter gene.

15. A method for assessing a test yeast for its maltose assimilation ability, comprising: culturing the test yeast; and measuring the expression level of the gene having the nucleotide sequence of SEQ ID NO: 1 and encoding a transcriptional inducer for maltase gene and maltose transporter gene.

16. A method for selecting a yeast, comprising: culturing test yeasts; quantifying the protein of claim 6 or measuring the expression level of the gene having the nucleotide sequence of SEQ ID NO: 1 and encoding a transcriptional inducer for maltase gene and maltose transporter gene; and selecting a test yeast having an amount of the protein or the gene expression level according to desired maltose assimilation ability.

17. The method for selecting a yeast according to claim 16, comprising: culturing a reference yeast and test yeasts; measuring for each yeast the expression level of the gene having the nucleotide sequence of SEQ ID NO: 1 and encoding a transcriptional inducer for maltase gene and maltose transporter gene; and selecting a test yeast having gene expression level higher than that in the reference yeast.

18. The method for selecting a yeast according to claim 16, comprising: culturing a reference yeast and test yeasts; quantifying the protein encoded by the polynucleotide in each yeast; and selecting a test yeast having a larger amount of the protein than that in the reference yeast.

19. A method for producing an alcoholic beverage comprising: conducting fermentation using the yeast according to claim 8 or a yeast selected by the method according to claim 16.


SEQUENCE LISTING (4 sequences)

SEQ ID NO: 1 - DNA, 1434 bp, Saccharomyces sp.
atgagtttcgttaagcaagcatgcgactgttgtcgcgttcgccgaataaagtgtgacggtaaaggaccgtgtagtcgctgccttcagcatgatttgaagtgtacataccttaaacagttaaagaaaagaggtccaaagtccatcagggctcgaagcttgaaaaaaatagccgatgtacagaaagctagtaatggtggcactacgatagctactgcggaagaatccacgaaggttccaaagaggctaatcgaccaatgcctaaggctttaccacaataatctgtacgttatatggcctctgctctgttacgaagatcttcacaggcttctggaagaaaggtatgatgactgttatgtgtactggtttctggtagctctctcggcagccactcttagtgacttgcaaactgaaatagaatctaaggagggagcttccttgactggaaaacagctatgtgacctttgtatgtcattacaccaccgttttgatgacctcaggggtagcgatgtttttagaataatgacgtattattatttgcatcgttgttttgctcaatcttctgatacaagaacttcgtacagactctcttgtgaagccattagcctcatcaagatcgccggatttcatcgcgaggaaacgtatgagcgtatctcgttcaatgagcagcagcttagacggaaggtgtattatttgcttcttttgactgaaagatattattccgtgtatatttactgcgcaacaagcctggatgccacgatatcaccaccgcaacctgaagttgtaactgaccctcgactttccttggatagtttcctcgagatgatcagagtatttactgtagcaggaaaatgtttttttgattcattagctgccaactccgtaaacgtttcttgtactgaagactccctcaaaaaaatatggagggaacttcatacaacgtcacttgaaatagagccatggtcgtacggatacatagacatttcattttcccggcattggatcagaacactggcttggaagctagcgcctctaacaaaaggtatacgaaccggtttcctttcaaacactaataatgcgctcataccagtcaaaattgccaaagacatgttggttgatacatttttaaccccaaaaagcttctatgaagttcacggccccggaataccaacaaaggcgctagaagtagcgaatgcgttggtagacgttgttatgaatcagttagaccagaatatgaaattggaggctttgaacgttttgcacgatatatccaaatttgttttttctttagaacactgtgatagtcggctgatcaagatatttaccgctaaatgtcaaactgcccttatttctatacctatttctagaccactagaattaagcgatgattctgaagaagacactaataaaattcttgaagaacatactgataagtga

SEQ ID NO: 2 - protein, 477 aa, Saccharomyces sp.
MSFVKQACDCCRVRRIKCDGKGPCSRCLQHDLKCTYLKQLKKRGPKSIRARSLKKIADVQKASNGGTTIATAEESTKVPKRLIDQCLRLYHNNLYVIWPLLCYEDLHRLLEERYDDCYVYWFLVALSAATLSDLQTEIESKEGASLTGKQLCDLCMSLHHRFDDLRGSDVFRIMTYYYLHRCFAQSSDTRTSYRLSCEAISLIKIAGFHREETYERISFNEQQLRRKVYYLLLLTERYYSVYIYCATSLDATISPPQPEVVTDPRLSLDSFLEMIRVFTVAGKCFFDSLAANSVNVSCTEDSLKKIWRELHTTSLEIEPWSYGYIDISFSRHWIRTLAWKLAPLTKGIRTGFLSNTNNALIPVKIAKDMLVDTFLTPKSFYEVHGPGIPTKALEVANALVDVVMNQLDQNMKLEALNVLHDISKFVFSLEHCDSRLIKIFTAKCQTALISIPISRPLELSDDSEEDTNKILEEHTDK

SEQ ID NO: 3 - DNA, 40 bp, Artificial
  Primer
gagctcatagcggccatgagtttcgttaagcaagcatgcg

SEQ ID NO: 4 - DNA, 42 bp, Artificial
  Primer
ggatcctatgcggccgcagcaaccaaaaagcttaaaaaactt